(19) United States
(12) Patent Application Publication
(10) Pub. No.: US 2023/0125562 A1
(43) Pub. Date: Apr. 27, 2023

(54) DECORATIVE WATER LAMP WITH AROMA DIFFUSER STRUCTURE

(71) Applicant: CHIN-SHENG YANG, Tainan City (TW)

(72) Inventor: CHIN-SHENG YANG, Tainan City (TW)

(21) Appl. No.: 17/510,400

(22) Filed: Oct. 26, 2021

(51) Int. Cl. F21S 10/00; A61L 9/01

(52) U.S. Cl. CPC F21S 10/002; A61L 9/01; A61L 2209/12; A61L 2209/15

(57) ABSTRACT

A decorative water lamp with an aroma diffuser structure includes: a water lamp body, having a transparent, three-dimensional shape and being an enclosed space, where the enclosed space is filled with a flowable liquid; a base, connected to and disposed at a bottom of the water lamp body, where an electronic control unit is disposed inside the base; and an incense holder, connected to the base and used for placing an incense body. In the present invention, the incense holder is disposed on one side of the base, to avoid occupying internal space of the base, to make the internal structure of the base simple and proper, and facilitate the deployment of the electronic control unit, a light-emitting component, a magnetic rotating shaft, and a power member.

### BACKGROUND

### Technical Field

The present invention relates to the technical field of decorative water lamps, and in particular, to a decorative water lamp with an aroma diffuser structure.

### Related Art

In daily life, some craft table decorations are often placed on indoor desktops and cabinets, and these decorations are nothing other than photo frames, toys, garage kits, and the like. However, all such decorations have only viewable static functions. Therefore, a crystal lamp decoration with internal sequins, glitter, or ornaments is designed in the current market, and the crystal lamp decoration is manually inverted or shaken or is electrified to make the internal sequins, glitter or ornaments produce a visual experience of liquid movement.

However, with the continuous development of society, there are an increasing quantity of decorative water lamps in a variety of styles on the market. However, changes of these decorative water lamps are merely external shape changes and some fixed or rotating decorations added inside the decorative water lamps, and can only be used for visual appreciation only, resulting in a relatively undiversified function. After years of experience in studying decorations such as table decorations, through trial and error and continuous improvement, the inventor conceives of and designs a decorative water lamp, where the decorative water lamp not only can be used for visual appreciation, but also can have a function of diffusing aroma of incense and be used for an olfactory experience, and has been granted the US patent “AROMA LAMP STRUCTURE, U.S. Pat. No. 8,158,066 B2” on record. Later, after years of experience in studying decorations such as table decorations, the inventor further conceives of and designs a novel aroma lamp structure, and therefore, creates the present model utility.

### SUMMARY

An objective of the present invention is to provide a decorative water lamp with an aroma diffuser structure having a simple structure, providing a visual experience and an olfactory experience, and having various functions.

To resolve the foregoing technical problems, the present invention may be implemented by using the following technical solutions:

A decorative water lamp with an aroma diffuser structure is provided, comprising:

a water lamp body, having a transparent, three-dimensional shape and comprising a hollow casing and a bottom cover, where the bottom cover is closed on a bottom of the hollow casing to form an enclosed space inside the hollow casing, and the enclosed space is filled with a flowable liquid;

a base, connected to and disposed at a bottom of the water lamp body, where an electronic control unit is disposed inside the base; and

an incense holder, connected to the base and used for placing an incense body.

In an embodiment, the incense holder is disposed on one side of the base, a trough comprising ventilating holes is provided at a top of the incense holder, a fan is disposed inside the incense holder and is connected to the electronic control unit, and air inlets in communication with the ventilating holes are provided at a bottom of the incense holder.

In an embodiment, the incense holder is pivotally disposed on the base, a trough comprising ventilating holes is provided at a top of the incense holder, a fan is disposed inside the base, and air inlets in communication with the ventilating holes are provided at an outer wall of the base.

In an embodiment, a magnetic moving element is disposed inside the water lamp body.

In an embodiment, the electronic control unit comprises at least one light- emitting component, the light-emitting component is electrically connected to the electronic control unit, the light-emitting component irradiates the water lamp body, a magnetic rotating shaft correspondingly attracted to the magnetic moving element is further disposed in the base, and the magnetic rotating shaft is driven by a power member to rotate.

In an embodiment, the magnetic rotating shaft is a transmission shaft and a magnetic member assembled corresponding to a top of transmission shaft.

In an embodiment, heightening pads are disposed at both a bottom of the base and the bottom of the incense holder.

In an embodiment, the light-emitting component is a light-emitting diode (LED).

Beneficial effects of the present invention are as follows:

1. Simple structure: In the decorative water lamp with an aroma diffuser structure in the present invention, the incense holder is disposed on one side of the base or pivotally disposed on the base, to facilitate the replacement of an incense body and better diffuse aroma of incense, and when the incense holder is disposed on one side of the base, the incense holder is prevented from occupying internal space of the base, to make the internal structure of the base simple and proper, and facilitate the deployment of the electronic control unit, the light-emitting component, the magnetic rotating shaft, and the power member. Finally, the overall structure of the decorative water lamp is simple, to facilitate production.

2. Diversified functions: By using the magnetic moving element, the liquid inside the water lamp body is enabled to flow, and in addition, the light-emitting component irradiates the water lamp body, and light is then refracted by the liquid, to produce a dazzling lighting effect, and in addition, the air quality of an environment space can be changed by diffusing aroma, to provide both a visual experience and an olfactory experience, so that the decorative water lamp is more versatile.

### BRIEF DESCRIPTION OF THE DRAWINGS

FIG. 1 is a schematic exploded view of Embodiment 1 of a decorative water lamp with an aroma diffuser structure according to the present invention;

FIG. 2 is a schematic diagram of an internal structure of Embodiment 1 of a decorative water lamp with an aroma diffuser structure according to the present invention;

FIG. 3 is a schematic structural diagram of Embodiment 1 of a decorative water lamp with an aroma diffuser structure according to the present invention;

FIG. 4 is a schematic exploded view of Embodiment 2 of a decorative water lamp with an aroma diffuser structure according to the present invention; and

FIG. 5 is a schematic diagram of an internal structure of Embodiment 2 of a decorative water lamp with an aroma diffuser structure according to the present invention.

As shown in the accompanying drawings: 100. water lamp body; 110. hollow casing; 120. bottom cover; 200. base; 300. incense holder; 310. ventilating hole; 320. trough; 330. fan; 340. air inlet; 400. magnetic moving element; 500. light-emitting component; 600. magnetic rotating shaft; 610. transmission shaft; 620. magnetic member; 700. power member; and 800. heightening pad.

### DETAILED DESCRIPTION

To make the foregoing objects, features, and advantages of the present invention more comprehensible, specific implementations of the present invention are described in detail below with reference to the accompanying drawings. In the following description, many specific details are described to give a full understanding of the present invention. The present invention can be implemented in many other manners different from those described herein. The term “and/or” used in this specification includes any or all combinations of one or more related listed items. A person skilled in the art may make similar improvements without departing from the connotation of the present invention. Therefore, the present invention is not limited to the specific embodiments disclosed below.

### Embodiment 1

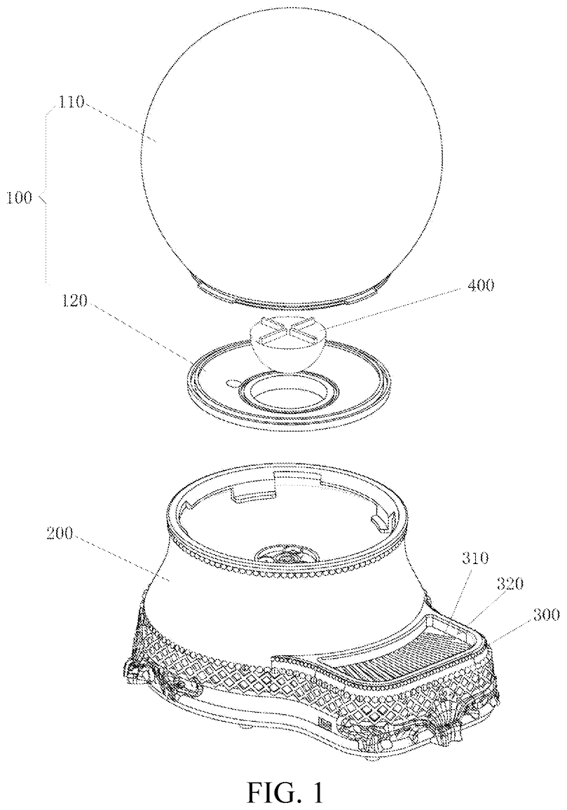

Referring to FIG. 1 to FIG. 3, a decorative water lamp with an aroma diffuser structure is provided, including:

a water lamp body 100, having a transparent, three-dimensional shape and including a hollow casing 110 and a bottom cover 120, where the bottom cover 120 is closed on a bottom of the hollow casing 110 to form an enclosed space inside the hollow casing 100, and the enclosed space is filled with a flowable liquid;

a base 200, connected to and disposed at a bottom of the water lamp body 100, where an electronic control unit is disposed inside the base 200; and

an incense holder 300, connected to the base 200 and used for placing an incense body.

The incense holder 300 is disposed on one side of the base 200, and is integrally connected to the base 200, a trough 320 including ventilating holes 310 is provided at a top of the incense holder 300, a fan 330 is disposed inside the incense holder 300 and is connected to the electronic control unit, and air inlets 340 in communication with the ventilating holes 310 are provided at a bottom of the incense holder 300, where the fan 330 is disposed between the air inlets 310 and the ventilating holes 340.

Specifically, in this embodiment, the water lamp body 100 has a transparent, three-dimensional shape, is hollow inside, and is filled with the liquid, a magnetic moving element 400 is further disposed inside the water lamp body 100, and the magnetic moving element 400 rotates to drive the liquid inside the water lamp body 100 to flow. In addition, the bottom of the water lamp body 100 and a top of the base 200 are installed, and the electronic control unit is disposed inside the base 200, where the electronic control unit includes a plurality of light-emitting components 500, and the light-emitting component 500 may be a light-emitting diode (LED). The electronic control unit makes the light-emitting component 500 emit light and irradiate the water lamp body 100, and the light is refracted by the liquid inside the water lamp body 100. Certainly, ornamental objects may be further disposed inside the water lamp body 100, and glitter may be added to the liquid. Finally, the refracted light is more dazzling and the lighting effect is more dazzling, to provide a better visual experience for users.

However, to improve the functionality of the decorative water lamp in this embodiment and make the structure of the decorative water lamp simple and proper, the incense holder 300 is disposed on one side of the base 200, the incense holder 300 may be integrally connected to the base 200, and the trough 320 having the ventilating holes 310 is provided at the top of the incense holder 300. By using the trough 320, the placement of the incense body can be more stable. The fan 330 is disposed inside the incense holder 300, and the air inlets 340 are provided at the bottom of the incense holder 300, where the fan 330 is disposed between the ventilating holes 310 and the air inlets 340, and certainly, is preferably disposed directly below the ventilating holes 310, to hydrodynamically maximize the circulation of a gas. In addition, the fan 330 is electrically connected to the electronic control unit, and the incense body is placed into the trough 320 at the top of the incense holder 300. The fan 330 then is started, the fan 330 rotates, and an air flow is transmitted from the air inlets 340 to the ventilating holes 310, to blow out aroma of the incense body in the trough 320, thereby changing the air quality of an environment space, so that the decorative water lamp provides an olfactory experience, and the decorative water lamp is more versatile. In addition, the incense holder 300 is disposed on one side of the base 200, to further prevent the incense holder 300 from occupying the internal space of the base 200, so that the internal structure of the base 200 is simple and proper. Finally, the overall structure of the decorative water lamp is simple, to facilitate production thereof.

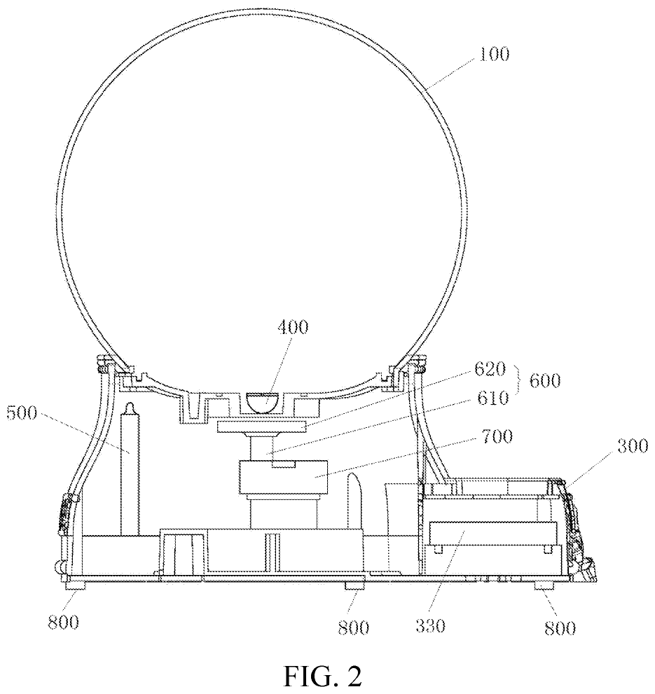

Referring to FIG. 2, to make the liquid inside the water lamp body 100 flow, the magnetic moving element 400 is disposed inside the water lamp body 100. To drive the magnetic moving element 400 to rotate to drive the liquid to flow, a magnetic rotating shaft 600 correspondingly attracted to the magnetic moving element 400 is further disposed inside the base 200, and the magnetic rotating shaft 600 is a transmission shaft 610 and a magnetic member 620 assembled corresponding to a top of transmission shaft 610. The transmission shaft 610 is connected to the power member 700, the power member 700 may be a motor, the motor drives the transmission shaft 610 to rotate, and the magnetic member 620 assembled with the top of the transmission shaft 610 rotates accordingly. Because the magnetic member 620 and the magnetic moving element 400 inside the water lamp body 100 have a magnetic attraction relationship, when rotating, the magnetic member 620 drives the magnetic moving element 400 to rotate accordingly, to drive the liquid inside the water lamp body 100 to flow, and when the liquid flows, the light-emitting component 500 irradiates the water lamp body 100 to enhance the dazzling visual effect of the decorative water lamp.

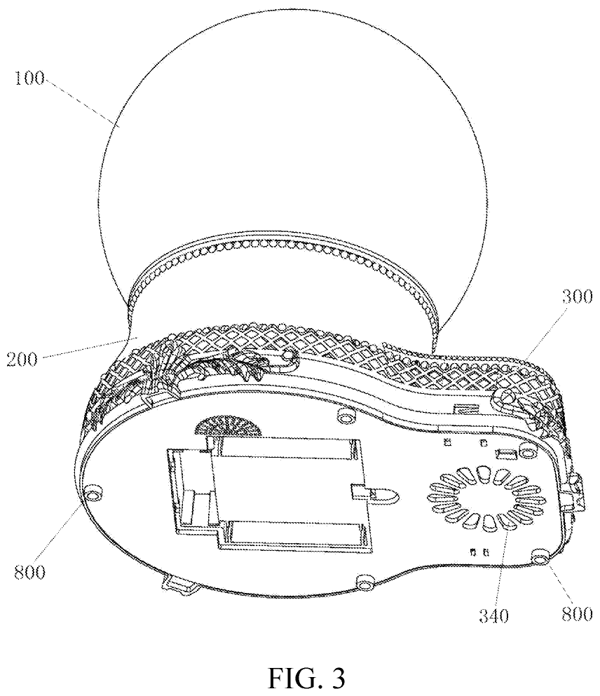

Referring to FIG. 2 or FIG. 3, because the gas needs to be circulated when the fan 320 inside the incense holder 300 runs, heightening pads 800 are disposed at both a bottom of the base 200 and the bottom of the incense holder 300. By using the heightening pads 800, after the decorative water lamp is placed on a platform such as a desktop or a cabinet panel, there is a specific gap between the bottom of the decorative water lamp and the platform, so that the gap allows the gas to easily enter from the air inlets 340 at the bottom of the incense holder 300, thereby circulating the gas and conveniently diffusing aroma of incense.

### Embodiment 2

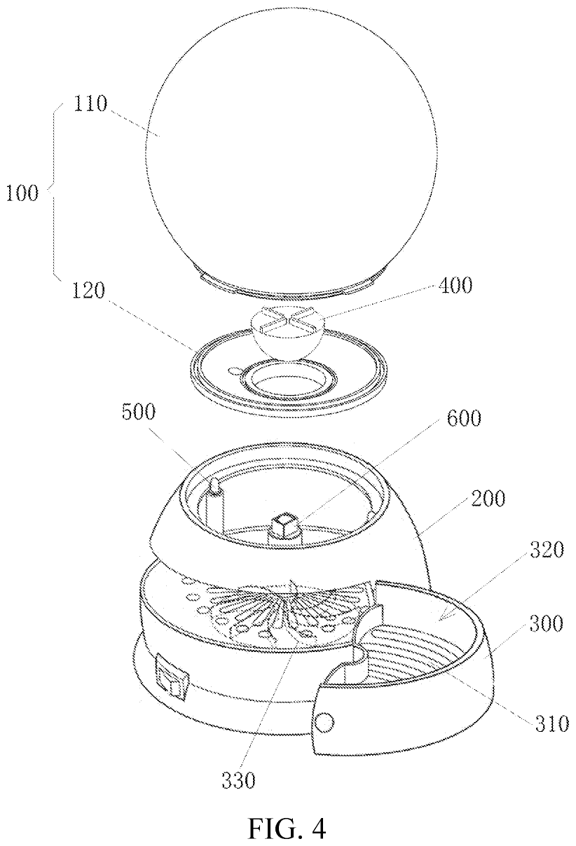

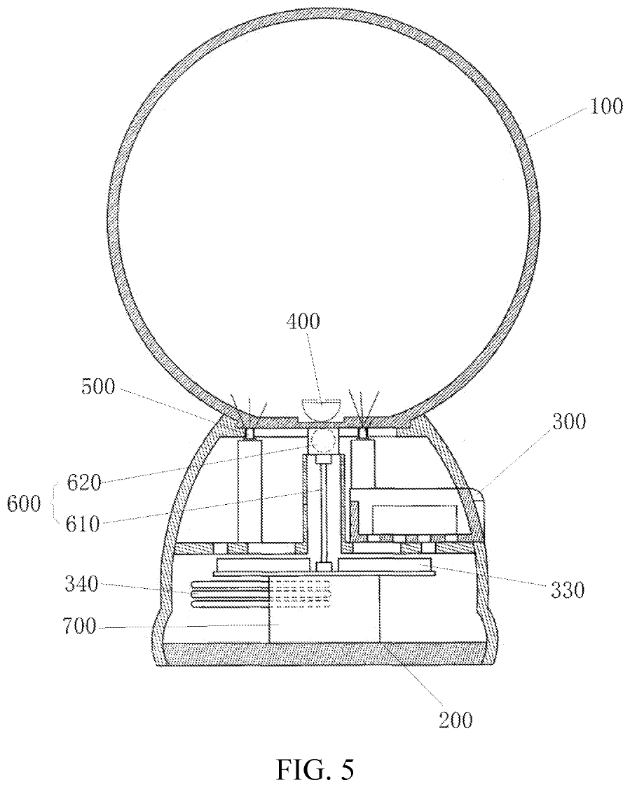

Referring to FIG. 4 and FIG. 5, in this embodiment, an incense holder 300 is pivotally disposed on a base 200, a trough 320 including ventilating holes 310 is provided at a top of the incense holder 300, a fan 330 is disposed inside the base 200, and air inlets 340 in communication with the ventilating holes 310 are provided at an outer wall of the base 200.

Specifically, in this embodiment, the incense holder 300 is pivotally disposed on the base 200, so that the incense holder 300 may be correspondingly screwed into the base 200 or screwed out of the base 200 to replace an incense body. In addition, a fan 330 is disposed inside the base 200, and the fan 330 is also disposed between the ventilating holes 310 and air inlets 340. The fan 330 rotates along with a magnetic rotating shaft 600 to blow out aroma of the incense body inside the trough 320. Similarly, to ensure the circulation of an air flow, the air inlets 340 are provided at the outer wall of the base 200. When the fan 330 rotates, the air flow may enter from the air inlets 340, and is then blown out from the ventilating holes 310 by the fan 330, so that the aroma of the incense body is blown out. In this way, the decorative water lamp in this embodiment provides both a visual experience and an olfactory experience. Finally, the decorative water lamp is more versatile.

The basic principles and main features of the present invention and the advantages of the present invention are shown and described above. Any person skilled in the art can smoothly implement the present invention according to the accompanying drawings and the foregoing description. However, some alterations, modifications, or evolutionary equivalent changes made by a person skilled in the art to the foregoing disclosed technical content without departing from the scope of the technical solutions of the present invention are all equivalent embodiments of the present invention. In addition, any alteration, modification, evolution, and the like of the equivalent changes made to the foregoing embodiments according to the essential technology of the present invention still fall within the protection scope of the technical solutions of the present invention.

What is claimed is:

1. A decorative water lamp with an aroma diffuser structure, comprising:
a water lamp body, having a transparent, three-dimensional shape and comprising a hollow casing and a bottom cover, wherein the bottom cover is closed on a bottom of the hollow casing to form an enclosed space inside the hollow casing, and the enclosed space is filled with a flowable liquid;
a base, connected to and disposed at a bottom of the water lamp body, wherein an electronic control unit is disposed inside the base; and
an incense holder, connected to the base and used for placing an incense body.

2. The decorative water lamp with an aroma diffuser structure according to claim 1, wherein the incense holder is disposed on one side of the base, a trough comprising ventilating holes is provided at a top of the incense holder, a fan is disposed inside the incense holder and is connected to the electronic control unit, and air inlets in communication with the ventilating holes are provided at a bottom of the incense holder.

3. The decorative water lamp with an aroma diffuser structure according to claim 1, wherein the incense holder is pivotally disposed on the base, a trough comprising ventilating holes is provided at a top of the incense holder, a fan is disposed inside the base, and air inlets in communication with the ventilating holes are provided at an outer wall of the base.

4. The decorative water lamp with an aroma diffuser structure according to claim 2, wherein a magnetic moving element is disposed inside the water lamp body.

5. The decorative water lamp with an aroma diffuser structure according to claim 3, wherein a magnetic moving element is disposed inside the water lamp body.

6. The decorative water lamp with an aroma diffuser structure according to claim 4, wherein the electronic control unit comprises at least one light-emitting component, the light-emitting component is electrically connected to the electronic control unit, the light-emitting component irradiates the water lamp body, a magnetic rotating shaft correspondingly attracted to the magnetic moving element is further disposed in the base, and the magnetic rotating shaft is driven by a power member to rotate.

7. The decorative water lamp with an aroma diffuser structure according to claim 5, wherein the electronic control unit comprises at least one light-emitting component, the light-emitting component is electrically connected to the electronic control unit, the light-emitting component irradiates the water lamp body, a magnetic rotating shaft correspondingly attracted to the magnetic moving element is further disposed in the base, and the magnetic rotating shaft is driven by a power member to rotate.

8. The decorative water lamp with an aroma diffuser structure according to claim 6, wherein the magnetic rotating shaft is a transmission shaft and a magnetic member assembled corresponding to a top of transmission shaft.

9. The decorative water lamp with an aroma diffuser structure according to claim 7, wherein the magnetic rotating shaft is a transmission shaft and a magnetic member assembled corresponding to a top of transmission shaft.

10. The decorative water lamp with an aroma diffuser structure according to claim 2, wherein heightening pads are disposed at both a bottom of the base and the bottom of the incense holder.

11. The decorative water lamp with an aroma diffuser structure according to claim 6, wherein the light-emitting component is a light-emitting diode (LED).

12. The decorative water lamp with an aroma diffuser structure according to claim 7, wherein the light-emitting component is a light-emitting diode (LED).
